(19) United States
(12) Patent Application Publication
(10) Pub. No.: US 2021/0177824 A1
(43) Pub. Date: Jun. 17, 2021

(54) EXTENDED RELEASE FORMULATIONS FOR INTRA-ARTICULAR APPLICATIONS

(71) Applicant: NOVARTIS AG, Basel (CH)

(72) Inventors: Carlo Castagnoli, Basel (CH); Andreas Fisch, Basel (CH); Mathilde Lorscheider, Saint Louis (FR); Manjali Laxman Neharkar, Thane (IN); Bernd Ulrich Riebesehl, Loerrach (DE)

(21) Appl. No.: 16/761,779

(22) PCT Filed: Nov. 8, 2018
(86) PCT No.: PCT/IB2018/058788

§ 371 Date: May 5, 2020

Related U.S. Application Data
(60) Provisional application No. 62/584,589, filed on Nov. 10, 2017.
(60) Provisional application No. 62/743,864, filed on Oct. 10, 2018.

(51) Int. Cl. A61K 31/4525; A61K 9/08; A61K 47/10; A61K 47/20; A61K 47/32; A61K 45/06; A61K 9/00

(52) U.S. Cl. CPC A61K 31/4525; A61K 9/08; A61K 47/10; A61K 9/0019; A61K 47/32; A61K 45/06; A61K 47/20

(57) ABSTRACT

The present invention relates to a pharmaceutical composition comprising an aqueous suspension of: (i) crystalline N-(3,4-dichlorophenyl)-3-(pyridin-4-yl)-7-oxabicyclo[2.2.1]heptane-2-carboxamide or a pharmaceutically acceptable salt thereof; and (ii) a surfactant comprising a water-soluble co-polymer characterized by >5% solubility in water at 25° C. The compositions provide extended release of N-(3,4-dichlorophenyl)-3-(pyridin-4-yl)-7-oxabicyclo[2.2.1] heptane-2-carboxamide, and are suitable for intra-articular injection to a joint of a patient suffering from arthritis, joint injury or cartilage injury.

### CROSS-REFERENCE TO RELATED APPLICATIONS

This application claims the benefit of U.S. provisional application Ser. No. 62/584,589 filed 10 Nov. 2017 and of U.S. provisional application Ser. No. 62/743,864 filed 10 Oct. 2018; each of which is incorporated by reference herein in its entirety.

### FIELD OF THE INVENTION

The present invention relates to extended release formulations for treating or preventing joint damage resulting from arthritis, joint injury or cartilage injury.

### BACKGROUND OF THE INVENTION

Arthritis is the inflammation of one of more joints, and affects approximately 350 million people worldwide. Osteoarthritis (OA) is the most common form of arthritis, and is characterized by a slow degenerative breakdown of the joint including both the articular cartilage and the subchondral bone underlying the articular cartilage. Joint damage (e.g., acute joint injury, such as a meniscal or ligament tear, or an intra-articular fracture) can also lead to arthritis, e.g., post-traumatic arthritis. Because articular cartilage has a limited ability to repair, even small undetectable damage can often get worse over time and lead to OA.

PCT/US15/30303, incorporated herein by reference in its entirety, describes N-(3,4-dichlorophenyl)-3-(pyridin-4-yl)-7-oxabicyclo[2.2.1] heptane-2-carboxamide (Compound A) and other compounds that are useful in preventing, ameliorating or treating arthritis and/or joint injury.

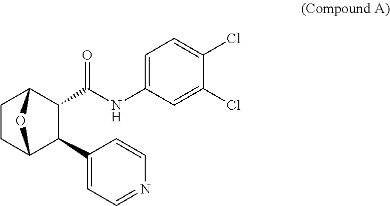 [SMILES: O=C(Nc1ccc(Cl)c(Cl)c1)[C@@H]1[C@@H](c2ccncc2)[C@@H]2CC[C@H]1O2]

Compound A is weakly basic with a pKa of 5.2, and is poorly soluble in neutral pH and basic pH aqueous solutions, but exhibits a strong pH dependent solubility. Solubility increases with decreasing pH. Compound A can be formulated as immediate release formulations for intra-articular injection. However, due to a high fluid exchange between the synovial fluid and the blood stream, immediate release formulations have a short synovial residence half-life and require frequent injections.

Thus, there remains a need for formulations that maintain efficacious levels of the drug substance in the synovial space for as long as possible, for treating chronic indications.

### SUMMARY OF THE INVENTION

The present invention provides extended release formulations comprising N-(3,4-dichlorophenyl)-3-(pyridin-4-yl)-7-oxabicyclo[2.2.1] heptane-2-carboxamide or a pharmaceutically acceptable salt thereof.

In one aspect, the present invention provides a pharmaceutical composition comprising an aqueous suspension of: (i) crystalline N-(3,4-dichlorophenyl)-3-(pyridin-4-yl)-7-oxabicyclo[2.2.1]heptane-2-carboxamide or a pharmaceutically acceptable salt thereof; and (ii) a surfactant comprising a water-soluble co-polymer characterized by >5% solubility in water at 25° C.

Various enumerated embodiments of the invention are described herein. Features specified in each embodiment may be combined with other specified features to provide further embodiments of the present invention. As used herein, % concentrations of ingredients in the compositions are in w/v %, unless otherwise stated.

Embodiment 1. A pharmaceutical composition as described above. In one embodiment, the composition is an injectable extended release formulation.

Embodiment 2. The pharmaceutical composition according to Embodiment 1, wherein said surfactant is a water-soluble co-polymer having a hydrophilic lipophilic balance (HLB) of at least 18.

Embodiment 3. The pharmaceutical composition according to Embodiment 1 or 2, wherein said surfactant is a water-soluble co-polymer having an average molecular weight between 7500-15000 Daltons.

Embodiment 4. The pharmaceutical composition according to Embodiment 1 or 2, wherein said surfactant is a water-soluble co-polymer having an average molecular weight between 8000-13000 Daltons.

Embodiment 5. The pharmaceutical composition according to any one of Embodiments 1-4, wherein said surfactant is a water-soluble co-polymer characterized by >5% solubility in water at 25° C. at 1 atm.

Embodiment 6. The pharmaceutical composition according to any one of Embodiments 1-4, wherein said surfactant is a water-soluble co-polymer characterized by >10% solubility in water at 25° C. at 1 atm.

Embodiment 7. The pharmaceutical composition according to any one of Embodiments 1-6, wherein said surfactant is a water-soluble block co-polymer, and optionally a water-soluble triblock co-polymer.

Embodiment 8. The pharmaceutical composition according to any one of Embodiments 1-7, wherein said surfactant is a water-soluble block co-polymer of formula

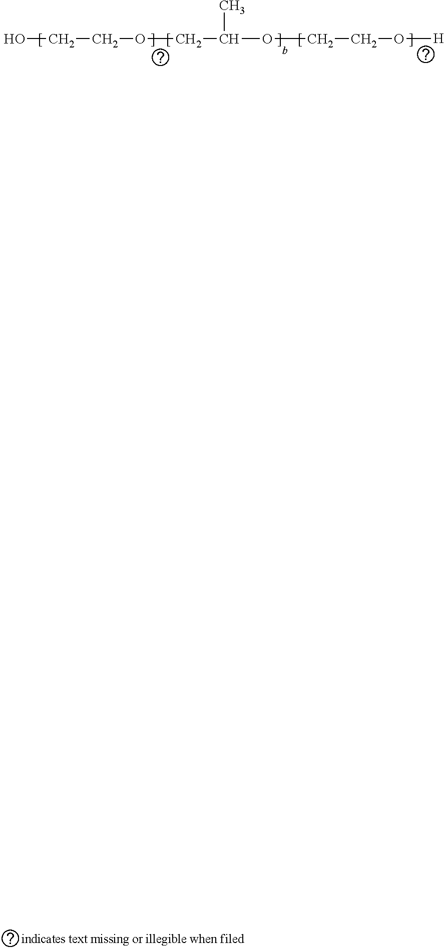

wherein:

a is 75-101; and

b is 25-60.

Embodiment 9. The pharmaceutical composition according to Embodiment 8, wherein said water-soluble block co-polymer is poloxamer 188.

Embodiment 10. The pharmaceutical composition according to Embodiment 8, wherein said water-soluble block co-polymer is poloxamer 407.

Embodiment 11. The pharmaceutical composition according to any one of Embodiments 8-10, wherein the concentration of said water-soluble block co-polymer is at least 0.025% w/v; for example, between 0.025-2% w/v, between 0.025-1% w/v, or preferably between 0.05-1% w/v.

Embodiment 12. The pharmaceutical composition according to Embodiment 1, wherein said surfactant further comprises sodium lauryl sulfate. In one embodiment, the concentration of said sodium lauryl sulfate is at least 0.05% w/v; more particularly, between 0.05-0.5% w/v.

Embodiment 13. The pharmaceutical composition according to any of Embodiments 1-12, further comprising a suspension stabilizer. In one embodiment, the concentration of said suspension stabilizer is between 0.1-10% w/v.

Embodiment 14. The pharmaceutical composition according to Embodiment 13, wherein said suspension stabilizer is selected from: (i) polyvinylpyrrolidone having an average molecular weight between 1-10 kDa, and optionally between 2-5 kDa; (ii) carboxymethyl cellulose having an average molecular weight between 25-2500 kDa, and optionally between 75-125 kDa; and (iii) a combination thereof.

Embodiment 15. The pharmaceutical composition according to Embodiment 14, wherein the concentration of said polyvinylpyrrolidone is between 1-5% w/v, and optionally between about 2-4% w/v; and the concentration of said carboxymethyl cellulose is 0.5-2% (w/v), and optionally between 0.75-1.5% w/v.

Embodiment 16. The pharmaceutical composition according to Embodiment 15 wherein said polyvinylpyrrolidone is PVP-12.

Embodiment 17. The pharmaceutical composition according to Embodiment 1, wherein said aqueous suspension comprises: (i) between 1-400 mg of crystalline N-(3,4-dichlorophenyl)-3-(pyridin-4-yl)-7-oxabicyclo[2.2.1] heptane-2-carboxamide per 1 mL of said aqueous suspension; (ii) between 0.05-1% w/v of water-soluble block co-polymer, optionally wherein said water-soluble block co-polymer is poloxamer 188 or poloxamer 407; and (iii) between 1-5% w/v of polyvinylpyrrolidone, optionally wherein said polyvinylpyrrolidone is PVP-K12.

Embodiment 18. The pharmaceutical composition according to Embodiment 17, comprising between 10-30 mg or about 25 mg of crystalline N-(3,4-dichlorophenyl)-3-(pyridin-4-yl)-7-oxabicyclo[2.2.1]heptane-2-carboxamide per 1 mL of said aqueous suspension.

Embodiment 19. The pharmaceutical composition according to Embodiment 18, comprising between 0.05-0.15% w/v, 0.08-0.12% w/v, or about 0.1% w/v of poloxamer 407.

Embodiment 20. The pharmaceutical composition according to Embodiment 17, comprising up to 75 mg, optionally between 40-60 mg or about 50 mg of crystalline N-(3,4-dichlorophenyl)-3-(pyridin-4-yl)-7-oxabicyclo[2.2.1]heptane-2-carboxamide per 1 mL of said aqueous suspension.

Embodiment 21. The pharmaceutical composition according to Embodiment 17, comprising between 80-120 mg or about 100 mg of crystalline N-(3,4-dichlorophenyl)-3-(pyridin-4-yl)-7-oxabicyclo[2.2.1]heptane-2-carboxamide per 1 mL of said aqueous suspension.

Embodiment 22. The pharmaceutical composition according to any one of Embodiments 20-21, comprising between 0.1-0.3% w/v or about 0.2% w/v poloxamer 407.

Embodiment 23. The pharmaceutical composition according to Embodiment 17, comprising between 150-250 mg or about 200 mg of crystalline N-(3,4-dichlorophenyl)-3-(pyridin-4-yl)-7-oxabicyclo[2.2.1]heptane-2-carboxamide per 1 mL of said aqueous suspension.

Embodiment 24. The pharmaceutical composition according to Embodiment 23, comprising between 0.1-0.5% w/v or about 0.2-0.4% w/v poloxamer 407.

Embodiment 25. The pharmaceutical composition according to Embodiment 17, comprising between 350-450 mg or about 400 mg of crystalline N-(3,4-dichlorophenyl)-3-(pyridin-4-yl)-7-oxabicyclo[2.2.1]heptane-2-carboxamide per 1 mL of said aqueous suspension.

Embodiment 26. The pharmaceutical composition according to Embodiment 25, comprising between 0.6-1% w/v, 0.7-0.9% w/v, or about 0.8% w/v poloxamer 407.

Embodiment 27. The pharmaceutical composition according to Embodiments 18 or 19, comprising between 1.5-2.5% w/v, or about 2% w/v PVP-K12.

Embodiment 28. The pharmaceutical composition according to any one of Embodiments 20-26, comprising between 1-5% w/v, or about 2-4% w/v PVP-K12.

Embodiment 29. The pharmaceutical composition according to Embodiment 1, wherein said aqueous suspension comprises: (i) between 1-100 mg of crystalline N-(3,4-dichlorophenyl)-3-(pyridin-4-yl)-7-oxabicyclo[2.2.1]heptane-2-carboxamide per 1 mL of said aqueous suspension; (ii) between 0.5-1.5 mg or about 1 mg poloxamer 407 per 1 mL of said aqueous suspension; and (iii) between 15-25 mg or about 20 mg PVP-K12 per 1 mL of said aqueous suspension.

Embodiment 30. The pharmaceutical composition according to Embodiment 29, comprising between 20-30 mg or about 25 mg of crystalline N-(3,4-dichlorophenyl)-3-(pyridin-4-yl)-7-oxabicyclo[2.2.1]heptane-2-carboxamide per 1 mL of said aqueous suspension.

Embodiment 31. The pharmaceutical composition according to any one of Embodiments 1-30, wherein said crystalline N-(3,4-dichlorophenyl)-3-(pyridin-4-yl)-7-oxabicyclo[2.2.1]heptane-2-carboxamide is microcrystalline or micronized.

Embodiment 32. The pharmaceutical composition according to any one of Embodiments 1-30, wherein said crystalline N-(3,4-dichlorophenyl)-3-(pyridin-4-yl)-7-oxabicyclo[2.2.1]heptane-2-carboxamide has a particle size distribution D90 of 50 microns or less, by laser light diffraction in suspension at a wavelength between 610-650 nm, and more particularly at about 630-635 nm.

Embodiment 33. The pharmaceutical composition according to any one of Embodiments 1-30, wherein said crystalline N-(3,4-dichlorophenyl)-3-(pyridin-4-yl)-7-oxabicyclo[2.2.1]heptane-2-carboxamide has a particle size distribution distribution D90 of 25 microns or less, by laser light diffraction in suspension, at a wavelength between 610-650 nm, and more particularly at about 630-635 nm.

Embodiment 34. The pharmaceutical composition according to one of Embodiments 1-33, wherein said N-(3,4-dichlorophenyl)-3-(pyridin-4-yl)-7-oxabicyclo[2.2.1]heptane-2-carboxamide is further defined as (1R,2R,3S,4S)—N-(3,4-dichlorophenyl)-3-(pyridin-4-yl)-7-oxabicyclo[2.2.1] heptane-2-carboxamide.

Embodiment 35. The pharmaceutical composition according to any one of Embodiments 1-34, further comprising a buffer capable of maintaining the pH of the suspension between 6 and 8.

Embodiment 36. The pharmaceutical composition according to any one of Embodiments 1-35, wherein said composition is suitable for intra-articular injection.

Embodiment 37. The pharmaceutical composition according to Embodiment 36, wherein said composition is suitable for intra-articular injection into a synovial cavity, particularly of the knee joint, in a patient suffering from arthritis, joint injury or cartilage injury. In one embodiment, the composition is suitable for intra-articular injection into the synovial cavity of a patient suffering from osteoarthritis. In another embodiment, the composition is suitable for intra-articular injection into the synovial cavity of a patient suffering from trauma arthritis. In another embodiment, the composition is suitable for intra-articular injection into the synovial cavity of a patient suffering from autoimmune arthritis.

Embodiment 38. The pharmaceutical composition according to Embodiment 37, wherein said composition provides extended release of N-(3,4-dichlorophenyl)-3-(pyridin-4-yl)-7-oxabicyclo[2.2.1]heptane-2-carboxamide into the synovial cavity for more than one hour, 24 hours, 7 days, 14 days or 30 days.

Embodiment 39. The pharmaceutical composition according to any one of Embodiments 1-35, wherein said composition is suitable for administration with a 22-31 gauge needle, a 29-31 gauge needle, or preferably a 30-gauge needle.

Embodiment 40. A combination comprising the pharmaceutical composition according to any one of Embodiments 1-35, and a second therapeutic agent.

Embodiment 41. A kit comprising a pharmaceutical composition according to any one of Embodiments 1-35, and at least instructions for use or a needle.

Embodiment 42. A pharmaceutical composition according to any one of Embodiments 1-35, and optionally in combination with a second therapeutic agent, for treating, ameliorating or preventing joint damage or injury, such as arthritis (osteoarthritis, trauma arthritis, or autoimmune arthritis such as systemic rheumatoid arthritis); degenerative disc disease; acute joint injury or cartilage injury.

Embodiment 43. A pharmaceutical composition according to any one of Embodiments 1-35 and optionally in combination with a second therapeutic agent, for inducing hyaline cartilage production, or for inducing differentiation of chondrocytes.

Embodiment 44. Use of a pharmaceutical composition according to any one of Embodiments 1-35, and optionally in combination with a second therapeutic agent, for the manufacture of a medicament for the treatment of joint damage or injury, such as arthritis (osteoarthritis, trauma arthritis, or autoimmune arthritis such as systemic rheumatoid arthritis); degenerative disc disease; acute joint injury or cartilage injury.

Embodiment 45. Use of a pharmaceutical composition according to any one of Embodiments 1-35, and optionally in combination with a second therapeutic agent, for the manufacture of a medicament for inducing hyaline cartilage production, or for inducing differentiation of chondrocytes.

Embodiment 46. A method for treating, ameliorating or preventing acute joint damage or injury in a subject in need thereof, comprising administering the pharmaceutical composition according to any one of Embodiments 1-35, and optionally in combination with a second therapeutic agent, to a subject in need thereof; thereby treating, ameliorating or preventing acute joint damage or injury in said subject.

Embodiment 47. The method according to Embodiment 46, wherein said pharmaceutical composition is administered to said subject in a dose range of up to 25 mg, up to 75 mg, up to 100 mg, up to 200 mg or up to 400 mg, of crystalline N-(3,4-dichlorophenyl)-3-(pyridin-4-yl)-7-oxabicyclo[2.2.1]heptane-2-carboxamide.

Embodiment 48. The method according to Embodiment 46 or Embodiment 47, comprising injecting the pharmaceutical composition into a synovial cavity of an individual having osteoarthritis.

Embodiment 49. A method of inducing hyaline cartilage production or differentiation of chondrocytes, comprising contacting chondrogenic progenitor cells with a therapeutically effective amount of the pharmaceutical composition according to any one of Embodiments 1-35, and optionally in combination with a second therapeutic agent; thereby inducing producing m hyaline cartilage extracellular matrix.

Embodiment 50. The method according to Embodiment 49, wherein said contacting step is performed in vitro or in vivo in a mammal; and when in vivo, stem cells are present in the mammal.

Embodiment 51. The method according to Embodiments 49 or 50, wherein said contacting step occurs in a matrix or biocompatible scaffold.

Embodiment 52. The pharmaceutical composition according to Embodiments 42 or 43, use according to Embodiments 44 or 45, or the methods according to any one of Embodiments 46-51, wherein said second therapeutic agent is selected from angiopoietin-like 3 protein (ANGPTL3), insulin growth factor (IGF1), SM04690, Janus kinase inhibitor, oral salmon calcitonin, SD-6010, vitamin D3, collagen hydrolysate, bone morphogenetic protein 7 (BMP7), rusalatide acetate, avocado soy unsaponifiables (ASU), a steroid, a non-steroidal anti-inflammatory agent (NSAID), hyaluronic acid, kartogenin, and TPX-100.

### BRIEF DESCRIPTION OF THE DRAWINGS

FIG. 1 (FIG. 1) is an electron microscope image of Compound A after three months at 40° C.

FIG. 2 (FIG. 2) compares the rat plasma concentration of Compound A after intra-articular administration of three extended release formulations of Compound A: microcrystal suspension, PLGA microparticle suspension, and MLV liposome suspension.

FIG. 3 (FIG. 3) compares the in vivo profile of a microcrystal extended release suspension of Compound A (250 μg) with an immediate release solution formulation (91 ng).

### DETAILED DESCRIPTION OF THE INVENTION

The present invention provides extended release formulations comprising a microcrystal suspension of N-(3,4-dichlorophenyl)-3-(pyridin-4-yl)-7-oxabicyclo[2.2.1] heptane-2-carboxamide or a pharmaceutically acceptable salt thereof, and a surfactant comprising a water-soluble co-polymer.

The present invention overcomes multiple challenges for preparing a microcrystal suspension of Compound A, including the identification of excipients that stabilize the drug suspension and provides acceptable features during manufacturing (compounding/filling) and administration (dosing accuracy, syringeability) of a highly concentrated crystal suspension (up to 100 mg/mL), including but not limited to the following:
Aspects for compounding:
   Quick suspendability of the drug substance (DS) (good wettability of the DS)
   Slow sedimentation of the initial suspension (prevention of aggregation of the DS microcrystals) in order to assure dosing precision during Good Manufacturing Practice (GMP) manufacturing
Aspects after autoclaving or storage:
   Chemical stability of the drug substance;
   Quick re-suspendability of micro-crystals
   Syringeability through 30 gauge needles to allow pain-less injection in the knee
   Slow sedimentation to allow dosing precision
   Absence of big crystals (Oswald ripening) or aggregates

### Definitions

As used herein, “extended release” refers to a dosage form that is deliberately modified to protract the release rate of the drug substance compared to that observed for an immediate-release dosage form. The release pattern in an extended release dosage may begin with a burst effect that mimics an immediate release, followed by a slower release of the remaining drug substance in the dosage form.

As used herein, the term “about” means within a statistically meaningful range of a value, typically within 10%. Such a range can lie within experimental error, typical of standard methods used for the measurement and/or determination of a given value or range. In one embodiment, the range is within 5% of the indicated value. In another embodiment, the range is within 1% of the indicated value. In yet another embodiment, the range is within 0.5% of the indicated value.

As used herein, the term “subject” refers to primates (e.g., humans, male or female), dogs, rabbits, guinea pigs, pigs, rats, mice and horses. In certain embodiments, the subject is a primate. In yet other embodiments, the subject is a human.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder

As used herein, a subject is “in need of” a treatment if such subject would benefit biologically, medically or in quality of life from such treatment.

As used herein, the term “a therapeutically effective amount” of a pharmaceutical composition refers to an amount of the composition that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to an amount of the Compound A extended release formulation that, when administered to a subject, is effective to (1) at least partially alleviate, inhibit, prevent and/or ameliorate joint damage resulting from joint injury and arthritis. In another non-limiting embodiment, the term “a therapeutically effective amount” refers to an amount of the Compound A extended release formulation that, when administered to a cell, or a tissue, or a non-cellular biological material, or a medium, is effective to promote chondrogenesis.

As used herein, the terms “treat”, “treating”, “treatment” plus “ameliorate” and “ameliorating” refer to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom or condition. The treatment or amelioration of symptoms can be based on any objective or subjective parameter; including, e.g., the result of a physical examination.

As used herein, “administering” refers to administration to a specific joint.

As used herein, the term “pharmaceutical composition” refers to a compound or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

As used herein, the term “a,” “an,” “the”, “said” and similar terms used in the context of the present invention (especially in the context of the claims) are to be construed to cover both the singular and plural unless otherwise indicated herein or clearly contradicted by the context.

The present invention provides extended release formulations comprising N-(3,4-dichlorophenyl)-3-(pyridin-4-yl)-7-oxabicyclo[2.2.1] heptane-2-carboxamide or a pharmaceutically acceptable salt thereof, particularly an injectable extended release formulation suitable for intra-articular injection to a joint of a patient suffering from arthritis, joint injury or cartilage injury.

In one aspect, the present invention provides a pharmaceutical composition comprising an aqueous suspension of: (i) crystalline N-(3,4-dichlorophenyl)-3-(pyridin-4-yl)-7-oxabicyclo[2.2.1]heptane-2-carboxamide or a pharmaceutically acceptable salt thereof; and (ii) a surfactant comprising a water-soluble co-polymer characterized by >5% solubility in water at 25° C.

In one embodiment, the pharmaceutical composition comprises a surfactant that is a water-soluble block co-polymer having a hydrophilic lipophilic balance (HLB) of at least 18. The hydrophilic-lipophilic balance (HLB) of a surfactant is a measure of the degree to which it is hydrophilic or lipophilic, and is determined by calculating values for the different regions of the molecule using methods known to those skilled in the art (e.g., BASF® PLURACARE® L/F Grades Poloxamer Technical Information, 04_070801e-1 Jul. 2009; BASF® Kolliphor® P Grades Technical Information, 03_111136e-03).

Examples of water-soluble block co-polymers, which may be suitable for use with the pharmaceutical compositions disclosed herein include but are not limited to poloxamer 188 (LUTROL® F-68), poloxamer 237 (PLURONIC® F-87), poloxamer 338 (PLURONIC® F-108) and poloxamer 407 (PLURONIC® F-127 or LUTROL® F-127), or a mixture thereof. The concentration of the poloxamer is generally about ≥0.025%; for example, between 0.025-2% w/v.

In another embodiment, the pharmaceutical composition may further comprise a suspension stabilizer, such as polyvinylpyrrolidine (PVP), carboxymethyl cellulose or a combination thereof. In particular embodiments, the additional suspension stabilizer is PVP-K12, alone or in combination with carboxymethyl cellulose.

In yet another embodiment, the pharmaceutical composition further comprises a suitable buffer capable of maintaining the pH of the aqueous suspension at physiologically acceptable pH between 6-8 or about 7.2-7.4. In one embodiment, the pharmaceutical composition comprises a phosphate buffer. Other known buffering agents, including but are not limited to, organic acid salts, TRIS or tromethamine hydrochloride, may be considered for use with the pharmaceutical compositions disclosed herein. In another embodiment, the pharmaceutical composition comprises NaCl for injection.

The pharmaceutical compositions of present invention may be administered simultaneously with, before or after, one or more other therapeutic agent(s). The pharmaceutical compositions of the present invention may be administered separately or together with one or more therapeutic agent, by the same or different routes of administration. A therapeutic agent is, for example, a chemical compound, peptide, antibody, antibody fragment or nucleic acid, which is therapeutically active or enhances the therapeutic activity when administered to a patient in combination with an extended release formulation of Compound A or a pharmaceutically salt thereof.

In one embodiment, the invention provides a product comprising an extended release formulation of Compound A or a pharmaceutically acceptable salt thereof, and at least one other therapeutic agent as a combined preparation for simultaneous, separate or sequential use in therapy. In one embodiment, the therapy is the treatment of joint damage resulting from joint injury or arthritis. Products provided as a combined preparation include a composition comprising an extended release formulation of Compound A or a pharmaceutically acceptable salt thereof, and the other therapeutic agent(s) together in the same pharmaceutical composition; or an extended release formulation of Compound A or a pharmaceutically acceptable salt thereof and the other therapeutic agent(s) in separate form, e.g. in the form of a kit.

In one embodiment, the invention provides an extended release formulation of Compound A or a pharmaceutically acceptable salt thereof in combination with a second therapeutic agent. The second agent may be one or more additional chondrocyte differentiation agent(s). Examples of chondrocyte differentiation agent include but are not limited to angiopoietin-like 3 protein (ANGPTL3), insulin growth factor (IGF1), SM04690 (Wnt inhibitor), Janus kinase inhibitors (such as ruxolitinib, tofacitinib, baricitinib), oral salmon calcitonin, SD-6010 (iNOS inhibitor), vitamin D3 (cholecalciferol), collagen hydrolyzate, bone morphogenetic protein 7 (BMP7), rusalatide acetate, avocado soy unsaponifiables (ASU), a steroid, a non-steroidal anti-inflammatory agent (NSAID), hyaluronic acid, kartogenin and TPX-100.

The pharmaceutical composition or combination of the present invention can be in a unit dosage of 0.5-1000 mg of active ingredient(s) for a subject of about 50-70 kg; for example, in a unit dosage range of 0.5-500 mg, 0.5-250 mg, 0.5-150 mg, 0.5-100 mg, or 0.5-50 mg of active ingredient(s). The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in in vitro and in vivo tests using advantageously mammals, e.g., mice, rats, dogs, monkeys or isolated organs, tissues and preparations thereof. The compositions of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

In one embodiment, the pharmaceutical composition of the present invention is administered intra-articularly in a dose range of up to 25 mg of active ingredient (e.g., Compound A); for example, about 0.5 mg, 2.5 mg, 7.5 mg, 15 mg or 25 mg of active ingredient. In another embodiment, the pharmaceutical composition of the present invention is administered intra-articularly in a dose range of up to 75 mg of active ingredient; for example, about 40 mg, 50 mg, 60 mg or 75 mg of active ingredient. In yet another embodiment, the pharmaceutical composition of the present invention is administered intra-articularly in a dose range of up to 100 mg of active ingredient; for example between 50-100 mg of active ingredient. In yet another embodiment, the pharmaceutical composition of the present invention is administered intra-articularly in a dose range of up to 200 mg or 400 mg of active ingredient.

In another aspect, the invention provides a kit comprising two or more separate pharmaceutical compositions, at least one of which contains an extended release formulation of Compound A or a pharmaceutically acceptable salt thereof. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compositions of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

### EXAMPLES

The following examples exemplify the invention, and does not limit the invention. One skilled in the art will understand that w/v % in the pharmaceutical compositions of the invention further includes acceptable variations within a statistically meaningful range and within acceptable experimental error, for example within 10% of the indicated value, and more preferably, within 5%, 1% or 0.5% of the indicated value.

### Abbreviations

The following abbreviations are used.
PVP-K12 Polyvinylpyrrolidone (Povidone K12); M.W. 3500 (CAS 9003-39-8)
CMC Carboxymethyl cellulose sodium salt
F68 LUTROL® F-68 (BASF)
F127 LUTROL® F-127 (BASF)
SDS Sodium dodecyl sulfate (sodium lauryl sulfate)
Egg PC L-α-phosphatidylcholine (natural lipids); MW 770.123 (average); (CAS 9281-44-2; Avanti Polar Lipids)
Phosphate buffer 50 nM, pH 7.0 (2.88 g of KH₂PO₄ and 4.099 g of Na₂HPO₄ in 1 L)
WFI Water for Injection

### Assays

Several assays were designed to test whether the formulations meet the criteria for intra-articular administration.

Suspendability. This assay measures the ability of the formulation to wet Compound A microcrystals and maintain the particles in suspension. Microcrystal suspensions of Compound A were stirred continuously for 5, 15, 30, and 60 minutes. At the end of the stirring period, each solution was evaluated according to the following criteria: (i) whether a homogenous suspension has been achieved; (ii) whether drug particles adhere to the vial glass surface; (iii) whether homogenous suspension has been achieved, but with residual cake adhering to the bottom; or (iv) whether the drug substance formed large agglomerates.

Sedimentation. This assay measures the sedimentation rate of the initial suspension. A slow sedimentation indicates less aggregation of the drug particles, ensuring better dosing precision. After the stirring step above, sample formulations were allowed to settle for 5, 15, 30 and 60 minutes. At the end of each period, each sample was evaluated following the same criteria for suspendability.

Syringeability. This assay measures whether a formulation can be quickly delivered to a joint without causing pain to the patient. A homogenous suspension was pulled up using a 30 gauge syringe, and re-injected back to the same vial. Syringeability was subjectively determined based on ease of pushing out the suspension from the needle.

### Example 1. Preparation of Micro-Crystals of Compound A

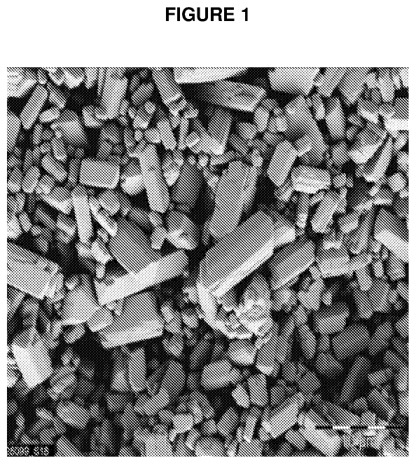

A production batch of Compound A was observed under the electronic microscope (FIG. 1). The particles were irregular and columnar, and ranged from about 100 nm up to 140 μm in length, but most of the particles are smaller than 50 μm. These particles have a tendency to agglomerate.

Compound A can be micronized using any methods known to those skilled in the art. Un-milled Compound A is subjected to a size reduction process to obtain particle size. The size reduction of Compound A can be performed using any known methods such as by jet-milling technology, and more particularly, by spiral jet-milling technology, fluidized bed opposed jet-milling technology or loop jet-milling technology.

In one embodiment, Compound A has a particle size distribution of D90≤50 μm. In another embodiment, Compound A has a particle size distribution of D90≤30 μm, ≤25 μm, ≤20 μm or ≤15 μm.

In yet other embodiments, Compound A was micronized to form micro-crystals with maximal particle diameters characterized in Table 1.

TABLE 1

| Parameter | Result |
|---|---|
| Surface area | 3 m²/g |
| Particle size: D10 | ≤1.4 μm |
| Particle size: D50 | ≤6.1 μm |
| Particle size: D90 | ≤29.7 μm |

### Example 2. Formulation Screening with PVP-K12 or CMC

Microcrystal suspensions of Compound A prepared in Example 1 were prepared with solutions containing PVP-K12 or carboxymethyl cellulose (CMC) sodium salt. The re-suspendability of the formulations was tested by repeating centrifugation and resuspension by shaking. The CMC containing formulations did not resuspend after centrifugation. PVP-K12 containing formulations were further investigated.

### Example 3. Formulations P1-P8 (PVP-K12 with F68, F127, SDS, or Egg PC)

Placebo solutions P1 to P8 were prepared according to the composition (w/v %) listed in Table 2.

TABLE 2

| | | Additional | Additional |
|---|---|---|---|
| Formulation | PVP-K12 (%) | excipient | excipient (%) |
| P1 | 2% | F68 | 0.2% |
| P2 | 2% | F68 | 0.5% |
| P3 | 2% | F127 | 0.2% |
| P4 | 2% | F127 | 0.5% |
| P5 | 2% | SDS | 0.05% |
| P6 | 2% | SDS | 0.01% |
| P7 | 2% | Egg PC | 0.2% |
| P8 | 2% | Egg PC | 0.5% |

Formulations were prepared in R2 vials with about 5 mg of Compound A microcrystals, and filled with placebo solutions P1 to P8 to reach a drug substance concentration of 5 mg/mL. The formulations were tested for re-suspendability and sedimentation rate according to the sequential steps listed in Column 1 of Table 3. After each step, the turbidity of the samples was observed and documented by photographs, and summarized in Table 3.

TABLE 3

| Step | P1 | P2 | P3 | P4 | P5 | P6 | P7 | P8 |
|---|---|---|---|---|---|---|---|---|
| Stirring | Turbid | Turbid | Turbid | Turbid | Turbid | Turbid | Turbid | Turbid |
| over night | | | | | | | | |
| Sedimentation 1 h | Partially | Partially | Partially | Partially | Turbid | Turbid | Turbid | Turbid |
| | clear | clear | clear | clear | | | | |
| Sedimentation 4 h | Clear | Clear | Partially | Partially | Turbid | Turbid | Turbid | Turbid |
| | | | clear | clear | | | | |
| 30 Gauge | OK | OK | OK | OK | OK | OK | OK | OK |
| Syringeability | | | | | | | | |
| after 15 min. | | | | | | | | |
| stirring | | | | | | | | |
| Centrifugation | Clear | Clear | Clear | Clear | Clear | Clear | Partially | Partially |
| 1 h (2000 g) | | | | | | | clear | clear |
| Resuspension | Possible | Possible | Possible | Possible | Possible | Possible | Possible | Possible |
| with hand | within | within | within | within | within | within | within | within |
| shaking | 30 sec | 30 sec | 30 sec | 30 sec | 1 min | 1 min | 1 min | 1 min |
| Heat sterilization | Clear | Clear | Partially | Partially | Turbid | Turbid | Turbid | Turbid |
| | | | clear | clear | | | | |
| Stirring | Turbid | Turbid | Turbid | Turbid | Turbid | Turbid | Turbid | Turbid |
| over night | | | | | | | | |
| Sedimentation 1 h | Partially | Partially | Partially | Partially | Turbid | Turbid | Turbid | Turbid |
| | clear | clear | clear | clear | | | | |
| Sedimentation 4 h | Clear | clear | Partially | Partially | Turbid | Turbid | Turbid | Turbid |
| | | | clear | clear | | | | |
| 30G | OK | OK | OK | OK | OK | OK | OK | OK |
| Syringeability | | | | | | | | |
| Centrifugation | Clear | Clear | Clear | Clear | Clear | Clear | Partially | Partially |
| 1 h (2000 g) | | | | | | | clear | clear |
| Resuspension | Possible | Possible | Possible | Possible | Possible | Possible | Possible | Possible |
| with hand | within | within | within | within | within | within | within | within |
| shaking | 30 sec | 30 sec | 30 sec | 30 sec | 1 min | 1 min | 1 min | 1 min |
| Light microscopy | No large | No large | No large | No large | No large | No large | No large | No large |
| after 15 min. | crystals | crystals | crystals | crystals | crystals | crystals | crystals | crystals |
| stirring | >50 μm | >50 μm | >50 μm | >50 μm | >50 μm | >50 μm | >50 μm | >50 μm |
| Resuspension | Possible | Possible | Possible | Possible | Not | Not | Not | Not |
| after 4 h | within | within | within | within | possible | possible | possible | possible |
| centrifugation | 1 min | 1 min | 1 min | 1 min | within | within | within | within |
| 4000 rpm (2000 g) | | | | | 3 min | 3 min | 3 min | 3 min |
| Resuspension | Possible | Possible | Possible | Possible | Not | Not | Not | Not |
| after 8 h | within | within | within | within | possible | possible | possible | possible |
| centrifugation | 3 min | 3 min | 5 min | 5 min | within | within | within | within |
| 4000 rpm (2000 g) | | | | | 3 min | 3 min | 3 min | 3 min |

The syringeability test provides a rough indication whether the formulation can be smoothly injected into a joint. For each vial, the suspension was withdrawn into a syringe through a 30 gauge needle and re-injected back to the same vial. The ease or difficulty of ejecting the content was categorized as “OK” or with difficulty.

Heat sterilization was performed at 122.5° C. and for 20 minutes. The suspendability and sedimentation of pre-heat sterilized and post-heat sterilized samples were observed to be the same. Thus, heat sterilization did not seem to have an effect on degradation and agglomeration of the microcrystals. Optical micrographs were taken from each resuspended formulations and screened for particles having an average particle size ≥50 μm; and no particles having an average particle size ≥50 μm were found in all formulations. The distribution of the micronized crystals was homogenous in all cases (micrographs not shown).

Formulations comprising SDS and egg PC (P5-P8) exhibited poor re-suspendability after 4 hrs of centrifugation. On the other hand, formulations comprising F68 and F127 (P1-P4) m exhibited better re-suspendability, and were further investigated.

### Example 4. Formulations P9-P16 (PVP-K12 with F68 or F127)

Placebo solutions P9 to P16 were prepared according to the composition (w/v %) listed in Table 4.

TABLE 4

| | | Additional | Additional |
|---|---|---|---|
| Formulation | PVP-K12 (%) | excipient | excipient (%) |
| P9 | 2% | F68 | 0.1% |
| P10 | 2% | F68 | 0.05% |
| P11 | 4% | F68 | 0.1% |
| P12 | 4% | F68 | 0.05% |
| P13 | 2% | F127 | 0.1% |
| P14 | 2% | F127 | 0.05% |
| P15 | 4% | F127 | 0.1% |
| P16 | 4% | F127 | 0.05% |

The formulations were prepared in R2 vials with about 5 mg of Compound A microcrystals added and filled up with placebo solutions P9 to P16 to reach a drug substance concentration of 5 mg/mL. The formulations were tested for re-suspendability and sedimentation rate according to the sequential steps listed in Column 1 of Table 5. After each step, the turbidity of the samples was observed and summarized in Table 5.

TABLE 5

| Step | P9 | P10 | P11 | P12 | P13 | P14 | P15 | P16 |
|---|---|---|---|---|---|---|---|---|
| Stirring 1 h | turbid | turbid | turbid | turbid | turbid | turbid | turbid | turbid |
| 30 G Syringeability | OK | OK | OK | OK | OK | OK | OK | OK |
| test | | | | | | | | |
| Resuspension after | Possible | Possible | Possible | Possible | Possible | Possible | Possible | Possible |
| 4 h centrifugation | within | within | within | within | within | within | within | within |
| 4000 rpm (2000 g) | 1 min | 1 min | 3 min | 3 min | 30 sec | 30 sec | 30 sec | 30 sec |
| 1 h stirring | Turbid | Turbid | Turbid | Turbid | Turbid | Turbid | Turbid | Turbid |
| 30 min sedimentation | Partially | Partially | Partially | Partially | Turbid | Partially | Partially | Partially |
| | clear | clear | clear | clear | | turbid | turbid | turbid |
| 1 h sedimentation | Almost | Almost | Almost | Almost | Turbid | Partially | Partially | Partially |
| | clear | clear | clear | clear | | clear | clear | clear |
| 2 h sedimentation | Clear | Clear | Clear | Clear | Turbid | Almost | Almost | Almost |
| | | | | | | clear | clear | clear |
| 4 h sedimentation | Clear | Clear | Clear | Clear | Turbid | Clear | Clear | Clear |
| 19 h sedimentation | Clear | Clear | Clear | Clear | Partially | Clear | Clear | Clear |
| | | | | | clear | | | |

Table 5 shows that all formulations containing F68 and F127 exhibited good resuspendability after a four-hour centrifugation. However, formulations containing F127 (P13-P16) were able to re-suspend the microcrystals within a shorter time and maintain the microcrystals in suspension longer when compared with formulations containing F68.

### Example 5. Formulations P17-P36 (PVP-K12 and F127)

Formulations P17 to P36, with or without F127, were designed as described in Table 6, with components listed as w/v %. As used herein, “Cpd A” refers to Compound A.

TABLE 6

| | PVP- | | | | | | | Phosphate | |
|---|---|---|---|---|---|---|---|---|---|
| | K12 | F127 | SDS | SDS | SDS | NaCl | Mannitol | 5 mM | Cpd A |
| No. | 2% | 0.05% | 0.01% | 0.003% | 0.001% | 0.875% | 5.45% | pH 7.0 | 5 mg/mL |
| P17 | x | x | | | | x | | x | x |
| P18 | x | | x | | | x | | x | x |
| P18A | x | x | x | | | x | | x | x |
| P19 | x | | | x | | x | | x | x |
| P20 | x | | | | x | x | | x | x |
| P21 | x | | | | | x | | x | x |
| P22 | x | x | | | | | x | x | x |
| P23 | x | | x | | | | x | x | x |
| P23A | x | x | x | | | | x | x | x |
| P24 | x | | | x | | | x | x | x |
| P25 | x | | | | x | | x | x | x |
| P26 | x | | | | | | x | x | x |
| P27 | | x | | | | x | | x | x |
| P28 | | | x | | | x | | x | x |
| P28A | | x | x | | | x | | x | x |
| P29 | | | | x | | x | | x | x |
| P30 | | | | | x | x | | x | x |
| P31 | | | | | | x | | x | x |
| P32 | | x | | | | | x | x | x |
| P33 | | | x | | | | x | x | x |
| P33A | | x | x | | | | x | x | x |
| P34 | | | | x | | | x | x | x |
| P35 | | | | | x | | x | x | x |
| P36 | | | | | | | x | x | x |

The above formulations P17 to P36 were prepared from their individual placebo solutions by adding in the target amounts of the microcrystals. The placebo solutions (vehicles) were prepared from stock solutions of each component. The components (in w/v %) and volume (in mL) of the component solutions needed to prepare a 50 mL placebo solution for each of the formulations, and the pH of the placebo solutions, were listed in Table 7.

TABLE 7

| | PVP- | | | NaCl | Mannitol | Phosphate | | pH |
|---|---|---|---|---|---|---|---|---|
| | K12 | F127 | SDS | 17.5 | 109 | 5 mM | WFI | Bulk |
| No. | 8.0% | 1.0% | 0.1% | mg/mL | mg/mL | pH 7.0 | (mL) | Placebo |
| P17 | 12.5 | 2.5 | | 25 | | 5 | 5 | 6.8 |
| P18 | 12.5 | | 5 | 25 | | 5 | 2.5 | 6.79 |
| P18A | 12.5 | 2.5 | 5 | 25 | | 5 | 0 | 6.79 |
| P19 | 12.5 | | 1.5 | 25 | | 5 | 6 | 6.84 |
| P20 | 12.5 | | 0.5 | 25 | | 5 | 7 | 6.85 |
| P21 | 12.5 | | | 25 | | 5 | 7.5 | 6.87 |
| P22 | 12.5 | 2.5 | | | 25 | 5 | 5 | 7.14 |
| P23 | 12.5 | | 5 | | 25 | 5 | 2.5 | 7.07 |
| P23A | 12.5 | 2.5 | 5 | | 25 | 5 | 0 | 7.06 |
| P24 | 12.5 | | 1.5 | | 25 | 5 | 6 | 7.03 |
| P25 | 12.5 | | 0.5 | | 25 | 5 | 7 | 7.05 |
| P26 | 12.5 | | | | 25 | 5 | 7.5 | 7.15 |
| P27 | | 2.5 | | | 25 | 5 | 17.5 | 6.95 |
| P28 | | | 5 | 25 | | 5 | 15 | 6.98 |
| P28A | | 2.5 | 5 | 25 | | 5 | 12.5 | 6.97 |
| P29 | | | 1.5 | 25 | | 5 | 18.5 | 6.94 |
| P30 | | | 0.5 | 25 | | 5 | 19.5 | 6.89 |
| P31 | | | | 25 | | 5 | 20 | 6.85 |
| P32 | | 2.5 | | | 25 | 5 | 17.5 | 7.06 |
| P33 | | | 5 | | 25 | 5 | 15 | 7.2 |
| P33A | | 2.5 | 5 | | 25 | 5 | 12.5 | 7.21 |
| P34 | | | 1.5 | | 25 | 5 | 18.5 | 7.21 |
| P35 | | | 0.5 | | 25 | 5 | 19.5 | 7.22 |
| P36 | | | | | 25 | 5 | 20 | 7.13 |

Table 8 describes the preparation of stock solutions for each excipient.

TABLE 8

| | |
|---|---|
| PVP-K12 8% | For 200 mL: weighed 16 g of |
| | PVP-K12 in 200 mL volumetric |
| | flask and completed with water. |
| F127 1% | For 50 mL: weighed 0.5 g of F127 |
| | in 50 mL volumetric flask |
| | and completed with water. |
| SDS 0.1% (5/50) | For 50 mL: weighed 0.05 g of SDS |
| | in 50 mL volumetric flask |
| | and completed with water. |
| SDS 0.1% (1.5/50) | For 10 mL: weighed 0.01 g of SDS |
| | in 10 mL volumetric flask |
| | and completed with water |
| SDS 0.1% (0.5/50) | For 5 mL: weighed 0.005 g of SDS |
| | in 5 mL volumetric flask |
| | and completed with water. |
| NaCl (17.5 mg/mL) | For 500 mL, weighed 8.75 g of NaCl |
| | in 500 mL volumetric |
| | flask and completed with water |
| Mannitol (109 mg/mL) | For 500 mL: weighed 54.5 g of |
| | Mannitol in 500 mL volumetric |
| | flask and completed with water |
| Phosphate buffer | For 1 L: weighed 2.88 g of KH₂PO₄ |
| 50 mM pH 7.0 | and 4.1 g of Na₂HPO₄ in 1 L volumetric |
| | flask and completed with water |

For each placebo solutions at 50 ml, the drug substance was added (5 mg/mL) and completed with the corresponding placebo by volume. Two vials of at least 1 mL of each formulation were prepared for subsequent assays. The first set of vials was assayed for their suspendability and sedimentation characteristics. The formulations were stirred continuously for 5, 15, 30, and 60 minutes. After 60 minutes of continued stirring, vials 17, 18, 18A, 19, 22, 23A, 27, 28 were homogeneous. The pH values measured at the completion of the stirring cycle compared well with those taken of the initial placebo solutions.

The formulations were tested under various assays to determine syringeability, sedimentation rate and suspendability. For syringeability, each sample formulation was withdrawn into a syringe through a 30 gauge needle and re-injected back to the same vial. All formulations had acceptable syringeability, even though the withdrawal of the samples was slow. Subsequently, the sample formulations were allowed to sediment for 60 minutes. After 60 minutes, vials 17, 18, 18A, 22, 23, 23A, 24, 27, 28A, 32 and 33A were still under suspension; vials 19, 20, 21, 25, 26 and 28 started to settle; and the rest of the vials had complete sedimentation. The vials were crimped shut and autoclaved at 122.5° C. for 20 minutes. All of the samples were clear, indicating that the microcrystals had sedimented. After hand shaking the vial a few times, only vials P17, P18, 23A, P27, and P28 re-suspended to homogenous suspensions. The second set of vials was subjected to five thermocycling cycles, each about 12 hours at 2-8° C. and 50° C., and evaluated as described above. The samples were hand shaken to re-suspend the particles, only vials P17, 18, 19, 27 and 28 were resuspended. However, most formulations (but not all) resuspended after more vigorous shaking. Syringeability was acceptable for all vials, except for P20 and P22.

Another sedimentation analysis was conducted. After 60 minutes, vials P17, P18A, P22, P23, P23A, P24, P27, P28A, P32 and P33A were still under suspension. The samples were transferred to Eppendorf tubes and centrifuged at 4000 rpm for 4 hours. Subsequently, the samples were shaken to resuspend. More than half resuspended after some vigorous shaking (vortexing), but only vials P17, P22, P27, P32 and P33A consistently resuspended after 60 minutes and after centrifugation.

### Example 6. Exemplary Extended Release Formulation

Example 6 provides an exemplary composition to illustrate but not limit the invention. The pharmaceutical compositions of the invention further includes acceptable variations known to one of ordinary skill in the art.

| Component | [mg/mL] |
|---|---|
| Compound A microcrystals | Up to 100 |
| PVP-K12 | 20 |
| Poloxamer 407 (LUTROL ® F127) | 1 |
| NaCl | 8.75 |
| Phosphate pH7 | 0.71 |

Surprisingly, the selected microcrystal suspension could be terminally sterilized by moist heat and did not show any signs of crystal growth (Oswald ripening) or aggregation during sterilization and over storage allowing accurate dosing through a thin needle (30 G). No local tissue irritation were observed. In one embodiment, the composition comprises 25 mg/mL Compound A microcrystals as a uniform suspension in the buffered vehicle above.

### Example 7. Extended Release Formulations Comparative Data

Several formulations with extended release characteristics were prepared and tested. The highest possible drug substance load was used in each formulation to maximize the therapeutic effect.

### Microcrystal Suspensions

Compound A (250 μg) was suspended in 25 μL of buffered vehicle from Example 6.

### PLGA Microparticle Suspension

Compound A (2% w/v) and PLGA (12 kDa; 1:1 L:G ratio) in dichloromethane was emulsified in an aqueous solution containing 1% w/v polyvinyl alcohol (PVA) to stabilize the initial emulsion. Evaporation of dichloromethane by heat resulted in Compound A molecularly dispersed in PLGA microparticles, which were subsequently washed in water and dried. PLGA microparticles having a particle size distribution of D50=40 μm and D90=57 μm were obtained.

### Liposome Suspension

A liposome formulation was used to solubilize the Compound A in a lipid bilayer and to obtain a possible sustained release. Additionally, the liposome should help as a lubricant at the injected side. A liposome formula was prepared as described in the procedures below:

### First Procedure

1. DMPC (100 mg) and unmicronized Compound A (3 mg) were dissolved in 10 mL EtOH:DCM 1:1
2. EtOH and DCM were removed on the rotavap for 30 min at 40° C. and 250 mbar (140 rpm)
3. Traces of EtOH and DCM were removed by 10 min at 40° C. and 40 mbar (140 rpm)
4. Lipid/Compound A film were rehydrated with 1 mL phosphate-buffered saline (PBS) or sugars in water
5. Samples where extruded through 1 um filter to obtain uniform sizes
6. Samples were frozen at −20° C.
7. Samples were lyophilized overnight
8. Samples were resuspended with sterile water

### Second Procedure

1. Compound A and DMPC where dissolved in tert-butanol
2. Samples were frozen at −20° C.
3. Samples were lyophilized overnight
4. Samples were resuspended with PBS

Original materials could be sterile filtered by solubilizing DMPC and Compound A in tert-butanol. After lyophilization, the powder could be reconstituted to manufacture multilamellar vesicles (MLV). The used lipids (DMPC) in its manufactured form (MLV) have the advantages of potentially better retention at the side of injection due to its large size and anti-inflammatory properties. Leakage of Compound A was tested in a mouse model, where a fast release of Compound A was observed.

### Comparative Data

Compound A was dosed intra-articularly (IA) in different extended release formulations to male Lewis Rats, 3 animal per formulation. The PLGA microparticle and MLV liposome suspensions were administered near the maximal possible dose, which may be limited by drug-loading capacity. Plasma samples were serial collected post IA injections up to 480 hours (20 days, n=3 per timepoint).

Plasma concentrations of Compound A were quantified using a Liquid Chromatography/Mass Spectrometry (LC/MS/MS) assay. Two hundred picogram per milliliter (pg/mL) of verapamil hydrochloride (Sigma-Aldrich, V4629, CAS 152-11-4) in acetonitrile/methanol, 3/1 by volume, was used as an internal standard and plasma precipitation solvent. To 20 μL of each plasma sample, 100 μL of internal standard solution was added to precipitate matrix proteins. The sample was vortexed then centrifuged with an Eppendorf Centrifuge 5810R (Eppendorf, Hamburg, Germany) at a setting of 4,000 rpm for 5 minutes at 10° C. The supernatant (80 μL) was transferred to a clean 96-well plate and mixed with 75 μL of Milli-Q water. The mixed samples were injected (5 μL) onto a ZORBAX® SB-C8 analytical column (2.1×30 mm, 3.5 μm, Agilent Technologies Inc., Palo Alto, Calif., USA) using a gradient method at flow rate of 700 μL/min (See Table below). Mobile phases consisting of 0.05% formic acid in water (solvent A) and 0.05% formic acid in acetonitrile (solvent B) were used. Compound A and internal standard were eluted at retention time 1.40 and 1.45 minutes, respectively.

HPLC Gradient

| Total Time (min) | A (%) | B (%) |
|---|---|---|
| 0.00 | 92 | 8 |
| 2.00 | 10 | 90 |
| 2.01 | 92 | 8 |
| 2.50 | 92 | 8 |

The HPLC system, consisting of Agilent 1260 series binary pump (Agilent Technologies Inc.), Agilent 1260 series micro vacuum degasser (Agilent Technologies Inc.), CTC PAL-HTC-xt analytics autosampler (LEAP Technologies, Carborro, N.C., USA) was interfaced to a Applied Biosystems SCIEX triple quadrupole 5500 mass spectrometer (AB Sciex LLC., Foster City, Calif., USA). Mass spectral analyses were carried out using electrospray ionization (ESI) in the positive ion mode. Compound A (363.03>156.00) and internal standard (455.40>165.10) peak integration were performed using Analyst™ 1.5 software. The lower limit of quantitation (LLOQ) in plasma was 5 pg/mL. Known amounts of Compound A were spiked into plasma to create quality control samples with known concentrations of 20, 80, 800, 4000 and 20000 pg/mL.

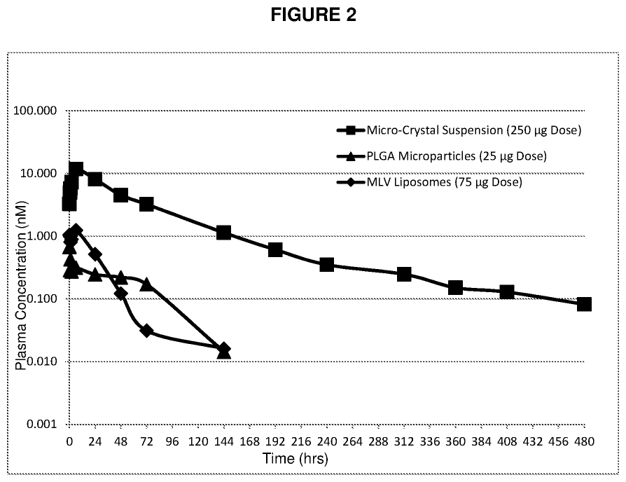

At time points up to 480 hours, rat plasma samples were collected and assayed for drug concentration. Table 9 shows the plasma pK parameters after intra-articular administration. As shown in FIG. 2 and Table 9, the microcrystal suspension outperformed the other formulations in enabling the highest drug dose, highest Cmax and drug exposure over time (AUC) until the end of the observation period. The microsuspension profile (▪) reaches the highest Cmax, since the drug load was the highest among the systems tested. Due to the longer residence time of microcrystal suspension of Compound A, the microcrystal suspension of Compound A was further profiled in vivo.

TABLE 9

| | | | | | | Dose | Dose |
|---|---|---|---|---|---|---|---|
| | | | | | | (normalized) | (normalized) |
| | Dose | T_{1/2} | Cₘₐₓ | Tₘₐₓ | AUC_{0-inf} | AUC_{0-inf} | Cmax |
| Formulation | [μg] | [hrs] | [nM] | [hrs] | [hrs*nM/μ] | [hrs*nM/μg] | [nM/μg] |
| Microcrystal | 250 | 102.4 | 11.8 | 6.0 | 761.9 | 3.0 | 0.047 |
| suspension | | | | | | | |
| PLGA microparticle | 25 | 43 | 0.74 | 0.69 | 26.4 | 1.1 | 0.029 |
| suspension | | | | | | | |
| MLV liposome | 75 | 18.8 | 1.9 | 2.2 | 33.1 | 0.44 | 0.025 |
| suspension | | | | | | | |

### Example 8. Immediate and Extended Release Formulation Comparative Data

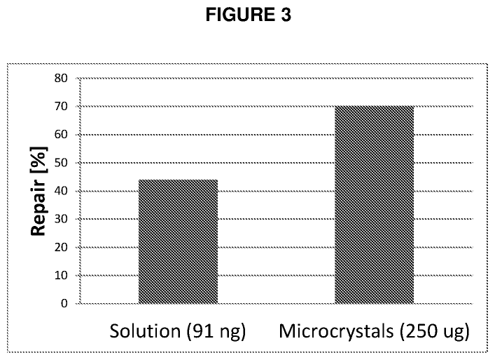

An in vivo study compared the immediate release (IR) and extended release (ER) formulations at deliverable dosages achievable with the respective formulation types in a rat meniscal tear (RMT) model in 36-week old Lewis males, following a single intra-articular injection after 4 weeks and take-down after 12 weeks (i.e., 8 weeks after injection). FIG. 3 compares the in vivo profiles of Compound A ER formulation (250 μg) and IR formulation (91 ng). As shown in FIG. 3, the extended release micro-crystal suspension was effective in repairing cartilage, and therefore provides the advantage of reduced dosing frequency (i.e., fewer injections) without compromising effectiveness of treatment.

### Example 9. Extended Release Formulations Comprising Compound a (25 Mg/mL)

Compound A (25 mg/mL) was dispersed in an aqueous buffered vehicle containing the following excipients, in the presence of anhydrous disodium phosphate (5 mM), NaOH (1N), HCl 25% (1N) as pH adjusting agents to set the pH to physiological pH 7, and water for injection.

| Vehicle | w/v (%) |
|---|---|
| PVP-K12 | 2% |
| Poloxamer 407 (LUTROL ® F127) | 0.1% |
| NaCl | .88% |

To increase the concentration of the drug substance, 50 mg of Compound A powder was added to 2 mL of vehicle solution as above. The solution was stirred overnight at 500 rpm with magnetic stirring, and a homogeneous suspension of individually dispersed microcrystals with no visible aggregates or agglomerates was obtained.

### Example 10. Extended Release Formulation Comprising Compound a (50-400 Mg/mL)

Compositions comprising essentially the same vehicle composition as in Example 10 with varying concentrations of PVP K12 and poloxamer 407 were screened to evaluate the impact of excipient concentration on sedimentation and re-suspension of compositions comprising Compound A at concentrations of about 50 mg/mL to about 400 mg/mL. The following compositions (1)-(4) were obtained as dis-agglomerated suspensions that were easily resuspended after sedimentation (e.g., during autoclaving or upon storage for several days).

| | (1) | (2) | (3) | | (4) |
|---|---|---|---|---|---|
| Compound A | 50 mg/mL | 100 mg/mL | 200 mg/mL | | 400 mg/mL |
| PVP-K12 | 2-4% (w/v) | 2-4% (w/v) | 2-4% | (w/v) | 2-4% (w/v) |
| Poloxamer 407 | 0.2% (w/v) | 0.2% (w/v) | 0.2%-0.4% | (w/v) | 0.8% (w/v) |

### Example 11. Carboxymethyl Cellulose as Additional Suspension Stabilizer

Carboxymethyl cellulose was added to a suspension comprising Compound A microcrystals (25 mg/mL) in the buffered vehicle of Example 6. Surprisingly, resuspension of non-autoclaved and auto-claved samples were improved in the presence of CMC (1% and 1.5% w/v).

It is understood that the examples and embodiments described herein are for illustrative purposes only. It is also understood that various modifications or changes in the described examples and embodiments will be suggested to persons skilled in the art, and are included within the spirit and purview of this application and the claims herein. All publications, patents, and patent applications cited herein are hereby incorporated by reference for all purposes.

1. A pharmaceutical composition comprising an aqueous suspension of: (i) crystalline N-(3,4-dichlorophenyl)-3-(pyridin-4-yl)-7-oxabicyclo[2.2.1]heptane-2-carboxamide or a pharmaceutically acceptable salt thereof; and (ii) a surfactant comprising a water-soluble co-polymer characterized by >5% solubility in water at 25° C.

2. The pharmaceutical composition of claim 1, wherein said surfactant is a water-soluble co-polymer having a hydrophilic lipophilic balance (HLB) of at least 18.

3. The pharmaceutical composition of claim 1, wherein said surfactant is a water-soluble co-polymer having an average molecular weight between 7500-15000 Daltons, or between 8000-13000 Daltons.

4. The pharmaceutical composition of claim 1, wherein said surfactant is a water-soluble co-polymer characterized by >10% solubility in water at 25° C.

5. The pharmaceutical composition of claim 1, wherein said surfactant is a water-soluble block co-polymer of formula 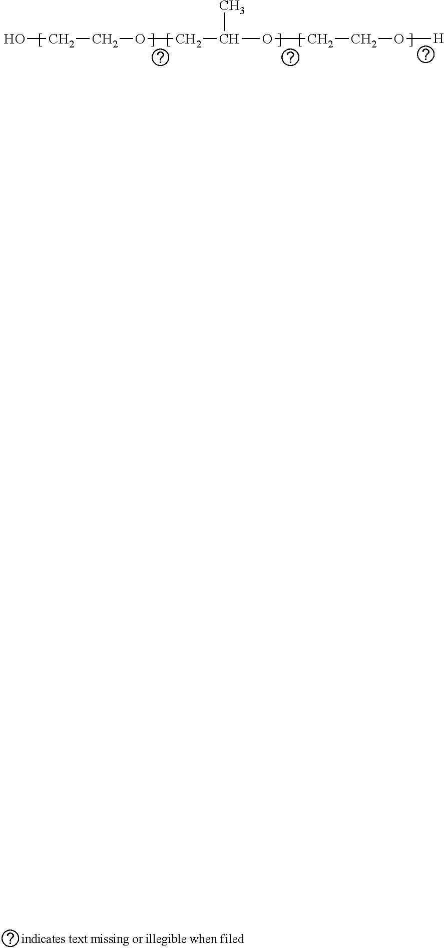
wherein:
a is 75-101; and
b is 25-60.

6. The pharmaceutical composition of claim 5, wherein said water-soluble block co-polymer is further defined as poloxamer 188.

7. The pharmaceutical composition of claim 5, wherein said water-soluble block co-polymer is further defined as poloxamer 407.

8. The pharmaceutical composition of claim 1, wherein the concentration of said water-soluble block co-polymer is at least 0.025% w/v, and optionally between 0.05-1% w/v.

9. The pharmaceutical composition of claim 1, wherein said surfactant further comprises sodium lauryl sulfate; and optionally wherein the concentration of said sodium lauryl sulfate is at least 0.05% w/v, and optionally between 0.05-0.5% w/v.

10. The pharmaceutical composition of claim 1, further comprising a suspension stabilizer; and optionally wherein the concentration of said suspension stabilizer is between 0.1-10% w/v.

11. The pharmaceutical composition of claim 10, wherein said suspension stabilizer is selected from: (i) polyvinylpyrrolidone having an average molecular weight between 1-10 kDa, and optionally between 2-5 kDa; (ii) carboxymethyl cellulose having an average molecular weight between 25-2500 kDa, and optionally between 75-125 kDa; and (iii) a combination thereof.

12. The pharmaceutical composition of claim 11, wherein the concentration of said polyvinylpyrrolidone is between 1-5% w/v, and optionally between about 2-4% w/v; and the concentration of said carboxymethyl cellulose is 0.5-2% (w/v), and optionally between 0.75-1.5% w/v.

13. The pharmaceutical composition of claim 12, wherein said polyvinylpyrrolidone is further defined as PVP-12.

14. The pharmaceutical composition of claim 1, wherein said aqueous suspension comprises: (i) between 1-400 mg of crystalline N-(3,4-dichlorophenyl)-3-(pyridin-4-yl)-7-oxabicyclo[2.2.1] heptane-2-carboxamide per 1 mL of said aqueous suspension; (ii) between 0.05-1% w/v of water-soluble block co-polymer, optionally wherein said water-soluble block co-polymer is poloxamer 188 or poloxamer 407; and optionally (iii) between 1-5% w/v of polyvinylpyrrolidone, optionally wherein said polyvinylpyrrolidone is PVP-K12.

15. The pharmaceutical composition of claim 1, comprising: (i) between 10-30 mg or about 25 mg of crystalline N-(3,4-dichlorophenyl)-3-(pyridin-4-yl)-7-oxabicyclo[2.2.1]heptane-2-carboxamide per 1 mL of said aqueous suspension; (ii) between 0.05-0.15% w/v or about 0.1% w/v of poloxamer 407; and optionally (iii) between 1.5-2.5% w/v or about 2% w/v PVP-K12.

16. The pharmaceutical composition of claim 1, comprising: (i) between 40-60 mg or about 50 mg, or between 80-120 mg or about 100 mg of crystalline N-(3,4-dichlorophenyl)-3-(pyridin-4-yl)-7-oxabicyclo[2.2.1]heptane-2-carboxamide per 1 mL of said aqueous suspension; (ii) between 0.1-0.3% w/v or about 0.2% w/v poloxamer 407; and optionally (iii) between 1-5% w/v or about 2-4% w/v PVP-K12.

17. The pharmaceutical composition of claim 1, comprising: (i) between 150-250 mg or about 200 mg of crystalline N-(3,4-dichlorophenyl)-3-(pyridin-4-yl)-7-oxabicyclo[2.2.1]heptane-2-carboxamide per 1 mL of said aqueous suspension; (ii) between 0.1-0.5% w/v or about 0.2-0.4% w/v poloxamer 407; and optionally (iii) between 1-5% w/v or about 2-4% w/v PVP-K12.

18. The pharmaceutical composition of claim 1, comprising: (i) between 350-450 mg or about 400 mg of crystalline N-(3,4-dichlorophenyl)-3-(pyridin-4-yl)-7-oxabicyclo[2.2.1]heptane-2-carboxamide per 1 mL of said aqueous suspension; (ii) between 0.6-1% w/v, 0.7-0.9% w/v, or about 0.8% w/v poloxamer 407; and optionally (iii) between 1-5% w/v or about 2-4% w/v PVP-K12.

19. The pharmaceutical composition of claim 1, wherein said crystalline N-(3,4-dichlorophenyl)-3-(pyridin-4-yl)-7-oxabicyclo[2.2.1]heptane-2-carboxamide is microcrystalline or micronized.

20. The pharmaceutical composition of claim 1, wherein said crystalline N-(3,4-dichlorophenyl)-3-(pyridin-4-yl)-7-oxabicyclo[2.2.1]heptane-2-carboxamide is crystalline (1R,2R,3S,4S)—N-(3,4-dichlorophenyl)-3-(pyridin-4-yl)-7-oxabicyclo[2.2.1] heptane-2-carboxamide.

21. The pharmaceutical composition of claim 1, further comprising a buffer capable of maintaining the pH of the suspension between 6 and 8.

22. The pharmaceutical composition of claim 1, wherein said composition is suitable for intra-articular injection.

23. A method for treating, ameliorating or preventing acute joint damage or injury in a subject in need thereof, comprising administering the pharmaceutical composition according to claim 1, and optionally in combination with a second therapeutic agent, to a subject in need thereof; thereby treating, ameliorating or preventing acute joint damage or injury in said subject.

24. The method of claim 23, wherein said pharmaceutical composition is administered to said subject in a dose range of up to 25 mg, up to 75 mg, up to 100 mg, up to 200 mg or up to 400 mg, of crystalline N-(3,4-dichlorophenyl)-3-(pyridin-4-yl)-7-oxabicyclo[2.2.1]heptane-2-carboxamide.

25. A combination comprising the pharmaceutical composition of claim 1, and a second therapeutic agent.

26. A kit comprising a pharmaceutical composition of claim 1, and at least instructions for use or a needle.
